(19) United States
(12) Patent Application Publication
(10) Pub. No.: US 2020/0138693 A1
(43) Pub. Date: May 7, 2020

(54) COMPOSITION FOR CONDITIONING HAIR

(71) Applicant: L'OREAL, Paris (FR)

(72) Inventors: Hui LIU, Shanghai (CN); Wi-Soon CHIA, Shanghai (CN); Maxime DE BONI, Shanghai (CN)

(73) Assignee: L'OREAL, Paris (FR)

(21) Appl. No.: 16/617,063

(22) PCT Filed: May 31, 2017
(86) PCT No.: PCT/CN2017/086576

§ 371 Date: Nov. 26, 2019

(51) Int. Cl. A61K 8/898; A61K 8/893; A61K 8/34; A61K 8/41; A61Q 5/12

(52) U.S. Cl. CPC A61K 8/898; A61K 8/893; A61Q 5/12; A61K 8/416; A61K 8/342

(57) ABSTRACT

Hair composition, especially for conditioning hair, is provided, which comprising: (i) at least one cationic surfactant of the formula (I), wherein: R₁ to R₄, identical or different, represent a linear or branched aliphatic group comprising from 1 to 30 carbon atoms, an aromatic group such as aryl or alkylaryl, at least one of the groups R₁ to R₄ comprising from 8 to 30 carbon atoms, and X⁻ is an anionic counterion chosen from halides, (ii) an oil-in-water emulsion having D50 particle size of less than 350 nm and comprising: a silicone mixture comprising (i) a trialkylsilyl terminated dialkylpolysiloxane having a viscosity of from 40,000 to less than 100,000 mPa·s at 25° C. and (ii) an amino-silicone having a viscosity of from 1,000 to 15,000 mPa·s at 25° C. and an amine value of from 2 to 10 mg of KOH per gram of amino-silicone, a mixture of emulsifiers comprising one or more nonionic emulsifiers, wherein the mixture of emulsifiers has a HLB value from 10 to 16, and water, and (iii) at least one fatty alcohol.

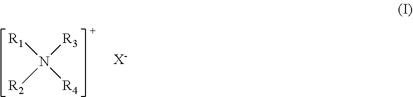

The present invention relates to hair compositions, such as compositions for caring for hair, more particularly for conditioning hair. The invention also relates to a cosmetic process for conditioning hair, using this composition.

In order to improve the cosmetic properties of the compositions for application to hair, in particular sensitized hair, i.e., hair which is damaged or weakened, in particular through the chemical action of environmental agents and/or of hair treatments such as permanent-waving, dyeing or bleaching, it is known to introduce into these compositions conditioning agents. The main purpose of these conditioning agents is to rectify or limit the undesirable effects induced by the various treatments or types of attach to which the hair fibers are more or less repeatedly subjected to and, of course, they can also improve the cosmetic behavior of natural hair.

The conditioning agents most commonly used to date are cationic polymers, silicones and/or silicone derivatives which impart to washed, dry or wet hair a disentangling, softness and a smoothness which are markedly enhanced in comparison to what can be obtained with corresponding compositions which do not contain them.

It is known, for example from U.S. Pat. No. 6,417,145, to use a mixture of silicone and cationic polymer in a shampoo, to wash and condition the hair. However, these compositions still have numerous disadvantages, such as presenting a low foam power and leading to an insufficient deposit of silicones on hair impacting therefore strongly on their cosmetic properties.

It is also known, for example from U.S. Pat. No. 6,610,280, a hair treatment composition containing a silicone component comprising droplets of silicone blend, said silicone blend comprising from 50 to 95% by weight of a first silicone having a viscosity of at least 100,000 mm²/sec and from 5 to 50% by weight a second silicone which is functionalized, for example amino-functionalized silicones.

But these compositions are not completely satisfactory. The silicone emulsions currently used in personal care compositions usually comprise high viscosity silicones that give a good smoothness and feel properties but have the disadvantage of giving heavy feel and build-up on the hair, and incur problem during removal from the hair while rinsing.

Also, the emulsions that are used in the prior art are mainly big blob emulsions and hence the silicone deposition is not very uniform, and the desired performance is not achieved.

Thus, there is a real need to provide cosmetic compositions, such as compositions for conditioning hair, that do not have the combination of drawbacks described above, i.e. which can enhance cosmetic properties of said fibers, namely by affording them softness, smoothness and disentangling.

The composition should trigger satisfactory silicone deposit on the keratin fibers.

The Applicant has now discovered that the use of a specific emulsion comprising silicones, especially in a conditioner base, makes it possible to achieve the objectives outlined above.

Thus, one object of the invention is especially a hair composition comprising:

(i) at least one cationic surfactant of the formula (I),

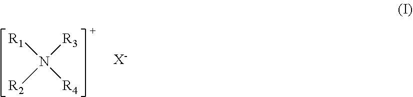

wherein:

R₁ to R₄, identical or different, represent a linear or branched aliphatic group comprising from 1 to 30 carbon atoms, an aromatic group such as aryl or alkylaryl,

at least one of the groups R₁ to R₄ comprising from 8 to 30 carbon atoms, and

X⁻ is an anionic counterion chosen from halides;

(ii) an oil-in-water emulsion having D₅₀ particle size of less than 350 nm and comprising:
a silicone mixture comprising (i) a trialkylsilyl terminated dialkylpolysiloxane having a viscosity of from 40,000 to less than 100,000 mPa·s at 25° C. and (ii) an amino-silicone having a viscosity of from 1,000 to 15,000 mPa·s at 25° C. and an amine value of from 2 to 10 mg of KOH per gram of amino-silicone;
a mixture of emulsifiers comprising one or more nonionic emulsifiers, wherein the mixture of emulsifiers has a HLB value from 10 to 16; and
water; and

(iii) at least one fatty alcohol.

Another object of the present invention is a process for the cosmetic treatment of hair, preferably for conditioning hair, comprising the steps of applying to the hair, preferably in a wet state, the hair composition described above, and optionally rinsing them with water after an optional period of exposure.

The composition according to the present invention is able to deposit a high amount of silicones on the hair, even when the composition contains a small amount of silicones.

In addition, the composition according to the invention leads to improved cosmetic properties, and especially affords good conditioning of the hair, including when the hair is sensitized. Indeed, the composition of the invention provides, for instance, smoothness, softness and disentangling to the hair.

In that which follows and unless otherwise indicated, the limits of a range of values are included within this range, in particular in the expressions “of between” and “ranging from . . . to . . . ” Moreover, the expression “at least one” or “at least a” used in the present description is equal to the expression “one or more”.

A—Surfactants

The composition of the present invention comprises at least one cationic surfactant, chosen from the compound of formula (I),

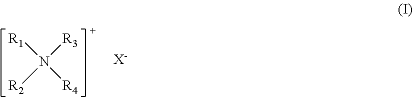

in which,

R₁ to R₄, which may be identical or different, represent a linear or branched aliphatic group comprising from 1 to 30 carbon atoms or an aromatic group such as aryl or alkylaryl, and at least one of the groups R₁ to R₄ comprising from 8 to 30 carbon atoms.

The aliphatic groups are chosen, for example, from C₁-C₃₀ alkyl, C₁-C₃₀ alkenyl, C₁-C₃₀ alkoxy, polyoxy(C₂-C₆)alkylene, C₁-C₃₀ alkylamide, (C₁₂-C₂₂)alkylamido(C₂-C₆)alkyl, (C₁₂-C₂₂)alkylacetate, C₁-C₃₀ hydroxyalkyl.

More preferably, at least one of the groups R₁ to R₄ comprises from 12 to 24 carbon atoms.

The aliphatic groups may comprise heteroatoms such as, in particular, oxygen, nitrogen, sulfur and halogens.

X⁻ is an anionic counterion chosen from halides.

Among the quaternary ammonium salts of formula (I), preference is given firstly to tetraalkylammonium halides such as tetraalkylammonium chlorides, for instance tetraalkylammonium or alkyltrimethylammonium halides such as dialkyldimethylammonium or alkyltrimethylammonium chlorides in which the alkyl group contains from approximately 12 to 22 carbon atoms, in particular halides such as behenyltrimethylammonium chloride, distearyldimethylammonium chloride, cetyltrimethylammonium chloride, benzyldimethylstearylammonium chloride, hydroxyethyl oleyldimethylammonium chloride, or else, and palmitylamidopropyltrimethylammonium halide, such as the chloride or stearamidopropyldimethyl(myristyl acetate)ammonium chloride.

Mentions may be made of the products, for example, sold under the name Dehyquart A OR by Cognis (INCI name: Cetrimonium chloride), products sold under the name Chimexane CL by the company Chimex (INCI name: hydroxyl oleyldimonium chloride), products sold under the name Fentacare 2232 EF by the company Rhodia (behenyltrimethylammonium chloride, with INCI name: behentrimonium chloride), or a mixture thereof.

More particularly, according to one embodiment of the invention, the cationic surfactant is present in an amount ranging from 0.01% to 10% by weight, preferably 0.05% to 5% by weight, relative to the total weight of the composition.

### B—Silicone-in-Water (or Oil-in-Water) Emulsion

The cosmetic composition according to the invention further comprises an oil-in-water emulsion having D₅₀ particle size of less than 350 nm and that comprises:
a silicone mixture comprising (i) a trialkylsilyl terminated dialkylpolysiloxane having a viscosity of from 40,000 to less than 100,000 mPa·s at 25° C. and (ii) an amino silicone having a viscosity of from 1,000 to 15,000 mPa·s at 25° C. and an amine value of from 2 to 10 mg of KOH per gram of amino silicone;
a mixture of emulsifiers comprising one or more nonionic emulsifiers, wherein the mixture of emulsifiers has a HLB value of from 10 to 16; and
water.

In the oil-in-water emulsion, or silicone-in-water emulsion, one liquid phase (the dispersed phase) is dispersed in the other liquid phase (the continuous phase); in the present invention, the silicone mixture, or silicone phase, is dispersed in the continuous aqueous phase.

The silicone mixture comprises one or more trialkylsilyl terminated dialkylpolysiloxanes that are preferably of formula (II):

R′₃SiO(R′₂SiO)ₚSiR′₃ formula (II)

wherein:
R′, same or different, is a monovalent hydrocarbon radical having from 1 to 18 carbon atoms, preferably from 1 to 6 carbon atoms, even better from 1 to 3 carbon atoms, more preferably methyl, and
p is an integer of from 500 to 2000, preferably of from 1000 to 2000.

The trialkylsilyl terminated (or end-blocked or α,ω-position) dialkylpolysiloxanes according to the invention have a viscosity of from 40,000 to less than 100,000 mPa·s (100,000 excluded) at 25° C., preferably a viscosity of from 40,000 to 70,000 mPa·s at 25° C., more preferably a viscosity of from 51,000 to 70,000 mPa·s at 25° C.

The trialkylsilyl terminated dialkylpolysiloxanes according to the invention are preferably linear but may contain additionally to the R′₂SiO_{2/2} units (D-units) in formula (II), RSiO_{3/2} units (T-units) and/or SiO_{4/2} units (Q-units), wherein R′, same or different, is a monovalent hydrocarbon radical having from 1 to 18 carbon atoms.

Preferably, R′, same or different, are alkyl radicals, preferably C₁-C₂₈ alkyl radicals, such as the methyl, ethyl, n-propyl, isopropyl, 1-n-butyl, 2-n-butyl, isobutyl, tert-butyl, n-pentyl, isopentyl, neopentyl and tert-pentyl radicals, hexyl radicals, such as the n-hexyl radical, heptyl radicals such as the n-heptyl radical, octyl radicals such as the n-octyl radical and isooctyl radicals, such as the 2,2,4-trimethylpentyl radical, nonyl radicals, such as the n-nonyl radicals, decyl radicals, such as the n-decyl radical, dodecyl radicals, such as the n-dodecyl radical, and octadecyl radicals, such as the n-octadecyl radical; alkenyl radicals such as the vinyl and ally radical; cycloalkyl radicals, such as the cyclopentyl, cyclohexyl, cycloheptyl and methylcyclohexyl radicals; aryl radicals, such as the phenyl, naphthyl, anthryl and phenanthryl radical; alkaryl radicals, such as the o-, m- and ptolyl radicals, xylyl radicals and ethylphenyl radicals; and aralkyl radicals such as the benzyl radical and the a- and the b-phenylethyl radical. Most preferred is the methyl radical.

Preferably, the trialkylsilyl terminated dialkylpolysiloxanes are trimethylsilyl terminated PDMS (polydimethylsiloxanes or dimethicones).

The silicone mixture comprises one or more amino silicones, that are preferably of formula (III):

XR₂Si(OSiAR)n(OSiR₂)mOSiR₂X formula (III)

wherein:
R, same or different, is a monovalent hydrocarbon radical having from 1 to 18 carbon atoms, preferably from 1 to 6 carbon atoms, even better from 1 to 3 carbon atoms, more preferably methyl,
X, same or different, is R or a hydroxyl (OH) or a C₁-C₆-alkoxy group; preferably X is R, i.e. a monovalent hydrocarbon radical having from 1 to 18 carbon atoms, preferably from 1 to 6 carbon atoms, even better from 1 to 3 carbon atoms, more preferably methyl, and
A is an amino radical of the formula —R′—[NR²—R³-]_{X}NR²₂, or the protonated amino forms of said amino radical, wherein R¹ is a C₁-C₆-alkylene radical, preferably a radical of the formula —CH₂CH₂CH₂— or —CH₂CH(CH₃)CH₂—, R², same or different, is a hydrogen atom or a C₁-C₄-alkyl radical, preferably a hydrogen atom, R³ is a C₁-C₆-alkylene radical, preferably a radical of the formula —CH₂CH₂—, and x is 0 or 1;
m+n is an integer from 50 to about 1000, preferably from 50 to 600.

Preferably, A is an amino radical of the formula —R′—[NR²—R³-]_{X}NR²₂, or the protonated amino forms of said amino radical, wherein R¹ is —CH₂CH₂CH₂— or —CH₂CH(CH₃)CH₂—, R² are hydrogen atoms, R³ is —CH₂CH₂—, and x is 1.

Preferably, R, same or different, are alkyl radicals, preferably C₁-C₂₈ alkyl radicals, such as the methyl, ethyl, n-propyl, isopropyl, 1-n-butyl, 2-n-butyl, isobutyl, tert-butyl, n-pentyl, isopentyl, neopentyl and tert-pentyl radicals, hexyl radicals, such as the n-hexyl radical, heptyl radicals such as the n-heptyl radical, octyl radicals such as the n-octyl radical and isooctyl radicals, such as the 2,2,4-trimethylpentyl radical, nonyl radicals, such as the n-nonyl radicals, decyl radicals, such as the n-decyl radical, dodecyl radicals, such as the n-dodecyl radical, and octadecyl radicals, such as the n-octadecyl radical; alkenyl radicals such as the vinyl and ally radical; cycloalkyl radicals, such as the cyclopentyl, cyclohexyl, cycloheptyl and methylcyclohexyl radicals; aryl radicals, such as the phenyl, naphthyl, anthryl and phenanthryl radical; alkaryl radicals, such as the o-, m- and ptolyl radicals, xylyl radicals and ethylphenyl radicals; and aralkyl radicals such as the benzyl radical and the a- and the b-phenylethyl radical. Most preferred is the methyl radical.

The amino silicones according to the invention have a viscosity of from 1,000 to 15,000 mPa·s at 25° C., preferably of from 1,500 to 15,000 mPa·s.

The amino silicones according to the invention have an amine value of from 2 to 10 mg of KOH per gram of amino silicone, preferably of from 3.5 to 8 mg.

The mole percent of amine functionality is preferably in the range of from about 0.3 to about 8%. Examples of amino silicones useful in the silicone mixture according to the invention include trialkylsilyl terminated amino silicone.

Most preferably, amino silicones are trimethylsilyl terminated aminoethylaminopropylmethylsiloxane, most preferably trimethylsilyl terminated aminoethylaminopropylmethylsiloxane—dimethylsiloxane copolymers.

The amino radical A can be protonated partially or fully by adding acids to the amino silicone, wherein the salt forms of the amino radical are obtained. Examples of acids are carboxylic acids with 3 to 18 carbon atoms which can be linear or branched, such as formic acid, acetic acid, propionic acid, butyric acid, pivalic acid, sorbic acid, benzoic acid, salicylic acid. The acids are preferably used in amounts of from 0.1 to 2.0 mol per 1 mol of amino radical A in the amino silicone of formula (III).

The silicone mixture preferably comprises (i) one or more trialkylsilyl terminated dialkylpolysiloxanes having a viscosity of from 40,000 to less than 100,000 mPa·s at 25° C. in a quantity of from 70 to 90% by weight, preferably from 75 to 85% by weight and (ii) one or more amino silicones having a viscosity of from 1,000 to 15,000 mPa·s at 25° C. and an amine value of from 2 to 10 mg of KOH per gram of amino silicone, in a quantity of from 10 to 30% by weight, preferably from 15 to 25% by weight, relative to the total weight of the silicone mixture.

The oil-in-water emulsion further comprises a mixture of emulsifiers that comprises one or more nonionic emulsifiers. It could optionally comprise one or more cationic surfactants.

The mixture of emulsifiers has a HLB value from 10 to 16.

The nonionic emulsifiers can be chosen among the nonionic surfactants previously described.

The nonionic emulsifiers could preferably be chosen among ethoxylated aliphatic alcohols, polyoxyethylene surfactants, carboxylic esters, polyethylene glycol esters, sorbitol ester and their ethoxylated derivatives, glycol esters of fatty acids, carboxylic amides, monoalkanolamine condensates, polyoxyethylene fatty acid amides.

Preferably, nonionic emulsifiers are selected from:

(i) polyoxyalkylene alkyl ethers, especially (poly)ethoxylated fatty alcohols of formula: R₃—(OCH₂CH₂)_{c}OH with:
R₃ representing a linear or branched C₈-C₄₀ alkyl or alkenyl group, preferably C₈-C₃₀ alkyl or alkenyl group, optionally substituted with one or more hydroxyl groups, and
c being an integer between 1 and 200 inclusive, preferentially between 2 and 150 and more particularly between 4 and 50, most preferably between 8 and 20.

The (poly)ethoxylated fatty alcohols are more particularly fatty alcohols comprising from 8 to 22 carbon atoms, oxyethylenated with 1 to 30 mol of ethylene oxide (1 to 30 OE);

(ii) polyoxyalkylene (C₈-C₃₂)alkylphenyl ethers,

(iii) polyoxyalkylene sorbitan (C₈-C₃₂) fatty acid esters, especially polyethoxylated fatty acid esters of sorbitan preferably containing from 2 to 40 ethylene oxide units, most preferably from 2 to 20 ethylene oxide units; preferably polyoxyethylenated sorbitan (C₁₀-C₂₄) fatty acid esters preferably containing from 2 to 40 ethylene oxide units, most preferably from 2 to 20 ethylene oxide units; and

(iv) polyoxyethylenated (C₈-C₃₂) fatty acid esters containing for example from 2 to 150 mol of ethylene oxide; preferably polyoxyethylenated (C₁₀—C₂₄) fatty acid esters containing for example from 2 to 150 mol of ethylene oxide.

Preferably, the nonionic emulsifiers could be selected from alkyl ether of polyalkyleneglycol and alkyl esters of polyalkyleneglycol; preferably of polyethyleneglycol.

Some useful emulsifiers are:
polyethyleneglycol octyl ether; polyethyleneglycol lauryl ether; polyethyleneglycol tridecyl ether; polyethyleneglycol cetyl ether; polyethyleneglycol stearyl ether; among these, mention may be made more particularly of trideceth-3, trideceth-10 and steareth-6.
polyethyleneglycol nonylphenyl ether; polyethyleneglycol dodecylphenyl ether; polyethyleneglycol cetylphenyl ether; polyethyleneglycol stearylphenyl ether;
polyethyleneglycol sorbitan monostearate, polyethyleneglycol sorbitan monooleate.
polyethyleneglycol stearate, and especially PEG 100 stearate.

Most preferably, the nonionic emulsifiers are chosen among steareth-6, PEG-100 stearate, trideceth-3 and trideceth-10 and their mixture; preferably, all these emulsifiers are present in the mixture of emulsifiers.

The mixture of emulsifiers could comprise one or more cationic emulsifiers that could be selected among tetraalkylammonium halides, tetraarylammonium halides, tetraalkylarylammonium halides, and their salts; quaternary ammonium compounds including salts; preferably, the cationic emulsifiers could be chosen among cetrimonium halides or behentrimonium halides, such as chloride.

The oil-in-water emulsion preferably comprises the mixture of emulsifiers in a total amount of from 5 to 15% by weight, preferably of from 8 to 15% by weight, most preferably of from 10 to 12% by weight, relative to the total weight of the emulsion.

The oil-in-water emulsion preferably comprises nonionic emulsifiers in a total amount of from 5 to 15% by weight, preferably of from 8 to 15% by weight, most preferably of from 10 to 12% by weight, relative to the total weight of the emulsion.

The oil-in-water emulsion preferably comprises cationic emulsifiers, when present, in a total amount of from 0.5 to 1.5% by weight, relative to the total weight of the emulsion.

The oil-in-water emulsion preferably comprises the silicone mixture in a total amount of from 40 to 60% by weight, preferably of from 45 to 55% by weight, relative to the total weight of the emulsion.

The oil-in-water emulsion preferably comprises the trialkylsilyl terminated dialkylpolysiloxane(s) in a total amount of from 35 to 45% by weight, preferably of from 38-42% by weight, relative to the total weight of the emulsion.

The oil-in-water emulsion preferably comprises the amino silicone(s) in a total amount of from 5 to 15% by weight, preferably of from 8-12% by weight, relative to the total weight of the emulsion.

The oil-in-water emulsion comprises water preferably in an amount of from 25 to 50% by weight, preferably of from 30 to 45% by weight, most preferably of from 35 to 42% by weight, relative to the total weight of the emulsion.

A method of preparation of the oil-in-water emulsion preferably comprises:
a step of mixing one or more trialkylsilyl terminated dialkylpolysiloxanes of viscosity of from 40,000 to less than 100,000 mPa·s at 25° C. and one or more amino silicones of viscosity of from 1,000 to 15,000 mPa·s at 25° C. and an amine value of from 2 to 10 mg of KOH per gram of amino silicone, at a temperature of from 15° C. to 40° C., preferably at 25° C., to obtain a mixed silicone fluid, then
a step of adding a mixture of emulsifiers comprising one or more nonionic emulsifiers, wherein the mixture of emulsifiers has a HLB value from 10 to 16, to the mixed silicone fluid to obtain a silicone-emulsifier-mixture, then
a step of homogenizing the silicone-emulsifier-mixture followed by
a step of adding, preferably step-wise, water, preferably demineralized water, to obtain an oil-in-water emulsion having D₅₀ particle size of less than 350 nm.

The preparation of the mixture of emulsifiers could be made by mixing one or more nonionic emulsifiers.

The pH of the oil-in-water emulsion after neutralization is preferably of from 4 to 6.

The oil-in-water emulsion has D₅₀ particle size of less than 350 nm, preferably of from 100 to 300 nm, more preferably from 150 to 250 nm, most preferably from 160 to 200 nm. It corresponds to the average hydrodynamic particle diameter. The D₅₀ particle size is expressed in volume. The D₅₀ particle size could be measured by using a device ZetaSizer from Malvern, UK, model Nano-ZS, which is based on the Photon Correlation Spectroscopy (PCS) method.

### Particle Size Measurement

Emulsion particle size is measured by using a device ZetaSizer from Malvern, UK, model Nano-ZS which is based on the Photon Correlation Spectroscopy (PCS) method. The D₅₀ value of particle size (average hydrodynamic particle diameter) is measured, wherein the evaluating algorithm is “cumulants analysis”.

Take 0.5 g of the emulsion sample in a 250 ml beaker, 100 ml of demineralized water is poured into it and then mixed properly to get the sample test solution. The sample test solution is poured in the cuvette cell and is put into the slot of the instrument to measure the particle size of the emulsion. D₅₀ is defined as the value of the particle diameter at 50% in the cumulative distribution. For example, if D₅₀=170 nm, then 50% of the particles in the sample are larger than 170 nm, and 50% smaller than 170 nm or about 50% by volume of all droplets in said emulsion is 170 nm.

### Viscosity Measurement

The viscosity, especially of the silicones or of the emulsion, is measured at 25° C.

For viscosities between 1000 to 40,000 mPa·s at 25° C.: the viscosity could be measured with an Anton Paar Rheometer; model MCR101, geometry single gap cylinder: CC27 spindle and shear rate of 1 s⁻¹ for 2 minutes, at 25° C.

For viscosities between 40,000 to 100,000 mPa·s at 25° C.: the viscosity could be measured with an Anton Paar Rheometer; model MCR101, 25-6 cone (Cone-plate geometry: 25 mm dia./6° cone); the “Zero gap” setting being made and with a shear rate of 1 s⁻¹ for 2 minutes, at 25° C.

Three measurements are made for each sample and the viscosity value is taken at 60 secs. MCR Rheometer Series products work as per USP (US Pharmacopeia Convention) 912—Rotational Rheometer methods.

### Amine Value Measurement

The amine value is determined by acid-base titration using a potentiometer [Make: Veego; Model: VPT-MG]. 0.6 g of sample is taken in a 500 ml beaker and a toluene-butanol 1:1 mixture is added and stirred to mix the sample thoroughly; then the sample solution is titrated with a 0.1(N) HCl solution. A determination of the blank value with the toluene-butanol 1:1 mixture is also done. The calculation of the amine value is done by the above mentioned potentiometer.

The amine value is calculated according to the formula:

56.11×(V−V_{Blank})N/W mg KOH/g of sample,

where V=Volume of HCl required in ml, V_{Blank}=Volume of HCl for blank value (without sample) with the toluene-butanol 1:1 mixture in ml; N=Normality of HCl, i.e. 0.1 N, W=weight of the sample taken in gram.

### HLB Value

The term HLB is well known to those skilled in the art, and denotes the hydrophilic-lipophilic balance of a surfactant or emulsifier. In the present invention, HLB values refer to the values at 25° C.

The HLB can be measured by experimental determination or can be calculated.

Calculation of HLB value of nonionic surfactants is calculated according to the equation: HLB=(E+P)/5, with E being the weight percentage of oxyethylene content and P being the weight percentage of polyhydric alcohol content, described in to the publication Griffin, J. Soc. Cosm. Chem. 1954 (vol. 5, no 4), pp. 249-256.

It can also experimentally be determined according to the book of F. Puisieux and M. Seiller, entitled “Galenica 5: Les systemes disperses—Tome I—Agents de surface et emulsions—Chapitre IV—Notions de HLB et de HLB critique, pp. 153-194—paragraph 1.1.2. Determination de HLB par voie experimentale [Experimental determination of HLB], pp. 164-180”.

The calculated HLB is the preferred HLB values that should be taken into account.

Said calculated HLB could be defined as being the following:

“calculated HLB=20×molar mass of the hydrophilic part/total molar mass.”

For an oxyethylenated fatty alcohol, the hydrophilic part corresponds to the oxyethylene units condensed onto the fatty alcohol and the “calculated HLB” then corresponds to the “Griffin HLB” as defined hereabove.

For an ester or an amide, the hydrophilic part is naturally defined as being beyond the carbonyl group, starting from the fatty chain(s).

For ionic surfactants/emulsifiers, the HLB value of individual surfactant/emulsifier can be calculated applying the Davies formula as described in Davies JT (1957), “A quantitative kinetic theory of emulsion type, I. Physical chemistry of the emulsifying agent”, Gas/Liquid and Liquid/Liquid Interface (Proceedings of the International Congress of Surface Activity): 426-438.

According to the formula, the HLB is derived by summing the hydrophilic/hydrophobic contribution afforded by the structural components of the emulsifier: HLB=(hydrophilic groups numbers)−n(group number per CH2 group)+7.

Approximate HLB values for some cationic emulsifiers are given in Table IV, in “Cationic emulsifiers in cosmetics”, GODFREY, J. Soc. Cosmetic Chemists (1966) 17, pp 17-27.

When two emulsifiers A and B of known HLB are blended for use, the HLB_{Mix} is said to be the required HLB for the mixture. This is expressed by the equation (W_{A}HLB_{A}+W_{B}HLB_{B})/(W_{A}+W_{B})=HLB_{Mix}, where W_{A}=the amount (weight) of the first emulsifier (A) used, and W_{B}=the amount (weight) of the second emulsifier (B); HLB_{A}, HLB_{B}=the assigned HLB values for emulsifiers A and B; HLB_{Mix}=the HLB of the mixture.

Advantageously, the composition according to the invention may comprise the oil-in-water emulsion in a quantity ranging of from 0.1% to 15% by weight, preferably from 0.5% to 10% by weight, more preferably from 1% to 8% by weight, even more preferably from 1.5% to 5% by weight, with respect to the total weight of the composition.

### C—Fatty Alcohol(s)

The composition of the present invention comprises at least one fatty alcohol.

The term “fatty alcohol” used herein refers to the compounds of formula R—OH (formula (IV)), which are linear or branched, saturated or unsaturated, and may contain from 8 to 40 carbon atoms.

Optionally, R maybe substituted with two or more hydroxyl groups.

Optionally, the fatty alcohol may be oxyalkylenated or glycerolated.

More preferably, in the fatty alcohol of formula (IV), R denotes from 8 to 30 carbon atoms; R preferably denotes a C₁₂-C₂₄ alkyl or a C₁₂-C₂₄ alkenyl group. Optionally, R may be substituted with one or more hydroxyl groups and especially with one or two hydroxyl groups.

Examples that may be mentioned include cetyl alcohol, stearyl alcohol, behenyl alcohol, isocetyl alcohol, isostearyl alcohol, isobehenyl alcohol and oleyl alcohol, and mixtures thereof.

The alcohol is preferably chosen from cetyl alcohol, stearyl alcohol, behenyl alcohol, or mixtures thereof.

Examples of the fatty alcohols may be mentioned are, for instance, cetyl alcohol that is sold under the name Lanette® 16 by the company BASF; stearyl alcohol that is sold under the name Lanette® 18 by the company BASF; behenyl alcohol that is sold under the name Lanette® 22 by the company BASF; the mixture of cetyl alcohol and stearyl alcohol, which is sold under the name Lanette® O OR by the company BASF.

The fatty alcohol(s) may be present in the composition in a content ranging from 1% to 15%, preferably from 2% to 10% and more preferably from 3% to 8% by weight relative to the total weight of the composition.

### D—Additional Silicone(s)

The composition according to the present invention preferably comprises one or more additional silicones, which is different from the silicone-in-water (or oil-in-water) emulsion as described above. Such silicones can be chosen in particular from non-amino silicones, amino silicones and mixtures thereof.

In the present invention, the term “silicone” is intended to denote, in accordance with what is generally accepted, any organosilicon polymer or oligomer of linear or cyclic, branched or crosslinked structure, of variable molecular weight, obtained by polymerization and/or polycondensation of suitably functionalized silanes, and consisting essentially of a repetition of main units in which the silicon atoms are linked together via oxygen atoms (siloxane bond —Si—O—Si—), optionally substituted hydrocarbon-based radicals being directly linked via a carbon atom to the said silicon atoms. The hydrocarbon-based radicals that are the most common are alkyl radicals, especially C₁-C₁₀ alkyl radicals, and in particular methyl, fluoroalkyl radicals, the alkyl part of which is C₁-C₁₀, and aryl radicals and in particular phenyl.

According to the present invention, the term “non-amino silicone” denotes any silicone not containing at least one primary, secondary or tertiary amine, or a quaternary ammonium group.

The non-amino silicones, which can be used in the composition according to the invention, are, in particular, polyorganosiloxanes that may be in the form of oils, waxes, resins or gums.

For the purposes of the present invention, the term “amino silicone” means any silicone comprising at least one primary, secondary or tertiary amine function or a quaternary ammonium group.

Preferably, the additional silicone that is suitable in the present invention is chosen from nonamino silicones.

Organopolysiloxanes are defined in greater detail in Walter Noll's Chemistry and Technology of Silicones (1968), Academic Press.

The silicones may be volatile or non-volatile. Preferably, the silicones are non-volatile silicones.

When the silicones are non-volatile, use is preferably made of polyalkylsiloxanes, polyarylsiloxanes, polyalkylarylsiloxanes, silicone gums and resins, and polyorganosiloxanes optionally modified with organofunctional groups, and mixtures thereof.

These silicones are more particularly chosen from polyalkylsiloxanes, among which mention may be made mainly of polydimethylsiloxanes containing trimethylsilyl end groups (Dimethicone according to the CTFA name) having a viscosity of from 5×10⁻⁶ to 2.5 m²/s at 25° C. and preferably 1×10⁻⁵ to 1 m²/s. The viscosity of the silicones is measured, for example, at 25° C. according to standard ASTM 445 Appendix C.

Among these polyalkylsiloxanes, mention may be made, in a non-limiting manner, of the following commercial products:
the Silbione oils of the 47 and 70 047 series or the Mirasil oils sold by the company Rhodia, for instance the oil 70 047 V 500 000,
the oils of the Mirasil series sold by the company Rhodia, in particular Mirasil® DM 500 000 sold by Rhodia.
the oils of the 200 series from the company Dow Corning, such as, more particularly, DC200 with a viscosity of 60 000 cSt,
the Viscasil oils from the company General Electric and certain oils of the SF series (SF 96, SF 18) from the company General Electric,

The polyalkylsiloxanes may also be in form of a silicone in water emulsion. Such products are available on the market, for example, emulsion of organopolysiloxane which contains 75% of dimethicone, sold by the company Shin-etsu under the name X52-2127.

Mention may also be made of polydimethylsiloxanes containing dimethylsilanol end groups (Dimethiconol according to the CTFA name) such as the oils of the 48 series from the company Rhodia.

Mention may also be made of polydimethylsiloxanes containing α,ω-silanol groups.

In this category of polyalkylsiloxanes, mention may also be made of the products sold under the names Abil Wax 9800 and 9801 by the company Goldschmidt, which are poly(C₁-C₂₀)alkylsiloxanes.

The polyalkylarylsiloxanes are particularly chosen from linear and/or branched polydimethylmethylphenylsiloxanes and polydimethyldiphenylsiloxanes with a viscosity of from 1×10⁻⁵ to 5×10⁻²m²/s at 25° C.

Among these polyalkylarylsiloxanes, examples that may be mentioned include the products sold under the following names:
Silbione oils of the 70 641 series from the company Rhodia,
the oils of the Rhodorsil 70 633 and 763 series from the company Rhodia,
the oil Dow Corning 556 Cosmetic Grade Fluid from the company Dow Corning,
silicones of the PK series from the company Bayer, such as the product PK20,
the silicones of the PN and PH series from the company Bayer, such as the products PN1000 and PH1000,
certain oils of the SF series from the company General Electric, such as SF 1023, SF 1154, SF 1250 and SF 1265.

The silicone gums that may be present in the composition according to the invention are especially polydiorganosiloxanes having high number-average molecular masses of between 200 000 and 1 000 000, preferably between 500 000 and 1 000 000, used alone or as a mixture in a solvent. This solvent can be chosen from volatile silicones, polydimethylsiloxane (PDMS) oils, polyphenylmethylsiloxane (PPMS) oils, isoparaffins, polyisobutylenes, methylene chloride, pentane, dodecane and tridecane, or mixtures thereof.

Mention may be made more particularly of the following products:
polydimethylsiloxane gums,
polydimethylsiloxane/methylvinylsiloxane gums,
polydimethylsiloxane/diphenylsiloxane gums,
polydimethylsiloxane/phenylmethylsiloxane gums,
polydimethylsiloxane/diphenylsiloxane/methylvinylsiloxane gums.

Products that may be used are the following:
polydimethylsiloxane sold by the company Bluestar under the name Mirasil DM 500 000, or by the company Dow Corning under the name Xiameter PMX⁻ 200 Silicone fluid (500 000 cSt),
mixtures formed from a polydimethylsiloxane hydroxylated at the end of the chain (known as dimethiconol according to the nomenclature of the CTFA dictionary) and from a cyclic polydimethylsiloxane (known as cyclomethicone according to the nomenclature of the CTFA dictionary), such as the product Q2 1401 sold by the company Dow Corning,
mixtures formed from a polydimethylsiloxane gum with a cyclic silicone, such as the product SF 1214 Silicone Fluid from the company General Electric, this product being an SF 30 gum corresponding to a dimethicone, having a number-average molecular weight of 500 000, dissolved in the oil SF 1202 Silicone Fluid corresponding to decamethylcyclopentasiloxane,
mixtures of two PDMSs with different viscosities, and more particularly of a PDMS gum and a PDMS oil, such as the product SF 1236 from the company General Electric. The product SF 1236 is a mixture of a gum SE 30 defined above, with a viscosity of 20 m²/s and of oil SF 96 with a viscosity of 5×10⁻⁶ m²/s. This product preferably comprises 15% of gum SE 30 and 85% of oil SF 96.

According to an embodiment, when exists, the additional silicone is present in an amount ranging from 0.01% to 10% by weight, preferably from 0.05% to 5% by weight, relative to the total weight of the composition.

### Others Ingredients

The composition of the present invention may comprise water, for example in a quantity of from 40 to 99% by weight, preferably from 50 to 98% by weight, most preferably from 55 to 95% by weight, relative to the total weight of the composition.

The composition according to the present invention may further comprise one or more additive(s) other than the compounds of the invention.

As additives that may be used in accordance with the invention, mention may be made of fatty substances, anionic, nonionic or amphoteric polymers or mixtures thereof, cationic surfactants, antidandruff agents, anti-seborrhoea agents, agents for preventing hair loss and/or for promoting hair regrowth, vitamins and provitamins including panthenol, sunscreens, mineral or organic pigments, sequestrants, plasticizers, solubilizers, acidifying agents, mineral or organic thickeners, especially polymeric thickeners, opacifiers, pearlescent or nacreous agents, antioxidants, hydroxyacids, fragrances and preserving agents.

According to a preferred embodiment, the present invention relates to a composition for conditioning hair, comprising, relative to the total weight of the composition:

(i) from 0.05% to 5% by weight of behenyltrimethylammonium chloride;

(ii) from 1.5% to 5% by weight of an oil-in-water emulsion having D₅₀ particle size of less than 350 nm and comprising:
the mixture of emulsifiers in a total amount of from 5 to 15% by weight, preferably of from 8 to 15% by weight, most preferably of from 10 to 12% by weight, relative to the total weight of the emulsion; and/or
nonionic emulsifiers in a total amount of from 5 to 15% by weight, preferably of from 8 to 15% by weight, most preferably of from 10 to 12% by weight, relative to the total weight of the emulsion; and/or
cationic emulsifiers in a total amount of from 0.5 to 1.5% by weight, relative to the total weight of the emulsion; and/or
the silicone mixture in a total amount of from 40 to 60% by weight, preferably of from 45 to 55% by weight, relative to the total weight of the emulsion; and/or
the trialkylsilyl terminated dialkylpolysiloxane(s) in a total amount of from 35 to 45% by weight, preferably of from 38-42% by weight, relative to the total weight of the emulsion; and/or
the amino silicone(s) in a total amount of from 5 to 15% by weight, preferably of from 8-12% by weight, relative to the total weight of the emulsion; and/or
water in an amount of from 25 to 50% by weight, preferably of from 30 to 45% by weight, most preferably of from 35 to 42% by weight, relative to the total weight of the emulsion; and

(iii) from 3% to 8% by weight of at least one fatty alcohol selected from the group consisting of cetyl alcohol, stearyl alcohol, or a mixture thereof.

Needless to say, a person skilled in the art will take care to select these optional additives such that the advantageous properties intrinsically associated with the composition according to the invention are not, or are not substantially, adversely affected by the envisaged addition(s).

The composition according to the invention may take the form of thickened liquid, creams or gel, They may also take the form of lotions.

The composition according to the invention is a hair composition; preferably it is a hair conditioning composition.

Another subject of the present invention is a process for the cosmetic treatment of hair, preferably for conditioning hair, comprising the steps of applying to the hair, preferably in a wet state, the composition described above, and optionally rinsing them with water after an optional leave-on time.

The leave-on time of the composition on hair may range from a few seconds to 15 minutes, better still from 5 seconds to 10 minutes and even better still from 10 seconds to 5 minutes.

The composition may be applied to wet or dry hair.

The examples that follow serve to illustrate the invention without, however, being limiting in nature.

The stability of the emulsion is determined after 3 months of storage at 45° C.; the stability is determined by no change in property of the emulsion; if the property changes, or if the oil and the aqueous phases separate, the emulsion is said to be unstable.

### EXAMPLE 1: PREPARATION OF THE OIL-IN-WATER EMULSION

Transfer 450 g of amino silicone fluid (trimethylsilyl terminated aminoethylaminopropylmethylsiloxane-dimethylsiloxane copolymer with amine value of 7.2 mg of KOH/gm sample, and a viscosity of 5600 mPa·s at 25° C.) in emulsion tank. Start stirring and under stirring condition transfer 1800 g of trimethylsilyl terminated dimethylsiloxane polymer fluid of viscosity 61,500 mPa·s at 25° C. in the same tank. Mix both the fluids for 2 hours at room temperature.

In a separate tank, transfer 49 g of steareth-6, 62 g of PEG100 stearate and heat to 60° C. Maintain the temperature till both emulsifiers become liquid. Then add 31 g of trideceth-3 and 350 g of trideceth-10 (80% of active matter). This nonionic emulsifiers mixture has an HLB value=11.25.

Then add 80 g water and 6.2 g glacial acetic acid in the tank and start mixing. Continue mixing till whole mass become a creamy paste. Whole paste is transfer to emulsion tank. Homogenize for 30 minutes at room temperature. Add 79.6 g demineralized water and homogenize for 60 minutes. Add 72.7 g demineralized water and homogenize for 50 minutes. Add 197.4 g demineralized water and homogenize for 5 minutes. Add 294.3 g demineralized water and homogenize for 5 minutes. Add 180 g demineralized water and homogenize for 5 minutes. Add 180 g demineralized water and homogenize for 5 minutes. Add 197.4 g demineralized water and homogenize for 5 minutes. Add 197.4 g demineralized water and homogenize for 3 minutes. Add 228.5 g demineralized water and homogenize for 3 minutes. Lastly add 40.5 g 2-phenoxyethanol as a biocide and homogenize for 3 minutes.

An stable oil-in-water emulsion having D₅₀ particle size of 170 nm is obtained.

### EXAMPLE 2: HAIR FORMULAS

The following formulas were prepared from the ingredients indicated in the table below, in which the amounts are given as mass percentages of active material relative to the total weight of the total amount of the formulas.

| | Invention formula | | | | Comparative formula | |
|---|---|---|---|---|---|---|
| | % by weight by active material | | | | | |
| Ingredient | 1 | 2 | 3 | 4 | 3′ | 2′ |
| Behentrimonium chloride (Fentacare ® 2232 EF from | 3.2 | 3.2 | 3.2 | 3.2 | 3.2 | 3.2 |
| Rhodia) | | | | | | |
| Cetearyl alcohol (Lanette ® O OR from BASF) | 5.5 | 5.5 | 5.5 | 7.5 | 5.5 | 0 |
| Cetyl Ester (Miraceti from Laserson) | 0.8 | 0.8 | 0.8 | 0.8 | 0.8 | 0.8 |
| Oil-in-water emulsion from example 1 | 0.6 | 1.2 | 1.2 | 3 | 0 | 1.2 |
| Dimethicone (X52-2127 from Shin-etsu, which contains | 1.2 | 1.2 | 0 | 0 | 0 | 1.2 |
| 75% of dimethicone, with a viscosity of 2000 to 10000 | | | | | | |
| mPa · s) | | | | | | |
| Dimethicone (Mirasil ® DM 500 000 by Bluestar) | 0.6 | 0.6 | 0 | 0 | 0 | 0.6 |
| Water | QS 100 | QS 100 | QS 100 | QS 100 | QS 100 | QS 100 |
| Fragrance | 0.5 | 0.5 | 0.5 | 0.5 | 0.5 | 0.5 |

Comparative formula 3′ (corresponding to the invention formula 3) does not contain the (ii) oil in water emulsion as claimed; comparative formula 2′ (corresponding to the invention formula 2) does not contain (iii) fatty alcohol as claimed.

The above mentioned formulas were prepared following a conventionally known formulation process for hair conditioning compositions.

### EXAMPLE 3: EVALUATION EXAMPLE

The invention and comparative formulas were applied on hair swatches and the coefficient of friction of the hair swatches were evaluated using the analyzing tool Coefficient of Friction MTT175 produced by the company Dia-Stron. The lower the coefficient of friction is, the better the cosmetic property of the formula.

The result is as follow:

Coefficient of Friction

| | |
|---|---|
| Invention formulas | Comparative formulas |

| 1 | 2 | 3 | 4 | 3′ | 2′ |
|---|---|---|---|---|---|
| 0.25 | 0.257 | 0.357 | 0.344 | 0.563 | Emulsion cannot |
| | | | | | be prepared |

According to the result obtained above, the invention formulas 1 to 4 present improved cosmetic properties and therefore affording improved hair softness, smoothness and disentangling, comparing to the comparative formula 3′.

Besides, the comparative formula 2′ is not a stable emulsion, whereas the invention formulas 1 to 4 are stable over time, i.e., stored for 2 months at temperature between 4° C. and 45° C.

Lastly, it is observed that the invention formulas 1 and 2 are the preferred formulas according to the invention.

1: A hair composition comprising:
(i) at least one cationic surfactant of the formula (I), 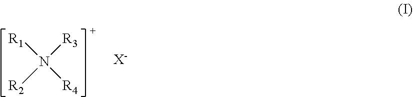
wherein:
R₁ to R₄, identical or different, represent a linear or branched aliphatic group comprising from 1 to 30 carbon atoms, an aromatic group such as aryl or alkylaryl,
at least one of the groups R₁ to R₄ comprising from 8 to 30 carbon atoms, and
X⁻ is an anionic counterion chosen from halides;
(ii) an oil-in-water emulsion having D₅₀ particle size of less than 350 nm and comprising:
a silicone mixture comprising (i) a trialkylsilyl terminated dialkylpolysiloxane having a viscosity of from 40,000 to less than 100,000 mPa·s at 25° C. and (ii) an amino-silicone having a viscosity of from 1,000 to 15,000 mPa·s at 25° C. and an amine value of from 2 to 10 mg of KOH per gram of amino-silicone;
a mixture of emulsifiers comprising one or more nonionic emulsifiers, wherein the mixture of emulsifiers has a HLB value from 10 to 16; and
water; and
(iii) at least one fatty alcohol.

2: The composition of claim 1, wherein in the formula (I), at least one of the groups R₁ to R₄ comprises from 12 to 24 carbon atoms, optionally comprising heteroatoms selected from the group consisting of oxygen, nitrogen, sulfur and halogens.

3: The composition of claim 1, wherein a content of the surfactant of formula (I) is from 0.01% to 10% by weight, relative to the total weight of the composition.

4: The composition according to claim 1, wherein the trialkylsilyl terminated dialkylpolysiloxane is of formula (II):
R′₃SiO(R′₂SiO)ₚSiR′₃ formula (II)
wherein:
R′, are each independently a monovalent hydrocarbon radical having from 1 to 18 carbon atoms, and
p is an integer of from 500 to 2000.

5: The composition according to claim 1, wherein the trialkylsilyl terminated dialkylpolysiloxane has a viscosity of from 40,000 to 70,000 mPa·s at 25° C.

6: The composition according to claim 1, wherein the amino silicones are of formula (III):
XR₂Si(OSiAR)ₙ(OSiR₂)ₘOSiR₂X formula (III)
wherein:
R are each independently a monovalent hydrocarbon radical having from 1 to 18 carbon atoms,
X are each independently R or a hydroxyl (OH) or a C₁-C₆-alkoxy group;
A is an amino radical of the formula —R¹—[NR²—R³-]_{X}NR²₂, or the protonated amino forms of said amino radical, wherein R¹ is a C₁-C₆-alkylene radical, R² are each independently a hydrogen atom or a C₁-C₄-alkyl radical, R³ is a C₁-C₆-alkylene radical, and x is 0 or 1;
m+n is an integer from 50 to about 1000.

7: The composition according to claim 1, wherein the amino silicones have a viscosity of from 1,500 to 15,000 mPa·s; and/or an amine value of from 3.5 to 8 mg of KOH per gram of amino silicone.

8: The composition according to claim 1, wherein the silicone mixture comprises (i) one or more trialkylsilyl terminated dialkylpolysiloxanes having a viscosity of from 40,000 to less than 100,000 mPa·s at 25° C. in a quantity of from 70 to 90% by weight, and (ii) one or more amino silicones having a viscosity of from 1,000 to 15,000 mPa·s at 25° C. and an amine value of from 2 to 10 mg of KOH per gram of amino silicone, in a quantity of from 10 to 30% by weight, relative to the total weight of the silicone mixture.

9: The composition according to claim 1, wherein the mixture of emulsifiers comprises one or more emulsifiers selected from the group consisting of:
(i) polyoxyalkylene alkyl ethers;
(ii) polyoxyalkylene (C₈-C₃₂)alkylphenyl ethers,
(iii) polyoxyalkylene sorbitan (C₈-C₃₂) fatty acid esters and
(iv) polyoxyethylenated (C₈-C₃₂) fatty acid esters containing from 2 to 150 mol of ethylene oxide.

10: The composition according to claim 1, wherein the oil-in-water emulsion comprises:
the mixture of emulsifiers in a total amount of from 5 to 15% by weight, relative to the total weight of the emulsion; and/or
nonionic emulsifiers in a total amount of from 5 to 15% by weight, relative to the total weight of the emulsion; and/or
cationic emulsifiers in a total amount of from 0.5 to 1.5% by weight, relative to the total weight of the emulsion; and/or
the silicone mixture in a total amount of from 40 to 60% by weight, relative to the total weight of the emulsion; and/or
the trialkylsilyl terminated dialkylpolysiloxane(s) in a total amount of from 35 to 45% by weight, relative to the total weight of the emulsion; and/or
the amino silicone(s) in a total amount of from 5 to 15% by weight, relative to the total weight of the emulsion; and/or
water in an amount of from 25 to 50% by weight relative to the total weight of the emulsion.

11: The composition according to claim 1, wherein the oil-in-water emulsion (ii) has a D₅₀ particle size of from 100 to 300 nm, expressed in volume.

12: The composition according to claim 1, wherein a content of the oil-in-water emulsion (ii) is from 0.1% to 15% by weight with respect to the total weight of the composition.

13: The composition according to claim 1, wherein the fatty alcohol is selected from the compound of formula (IV):
R—OH formula (IV)
wherein:
R denotes an alkyl or alkenyl group having from 8 to 30 carbon atoms, wherein R is optionally substituted with one or more hydroxyl groups.

14: The composition according to claim 1, wherein a content of the fatty alcohol is from 1% to 15%, by weight relative to the total weight of the composition.

15: The composition according to claim 1, further comprising at least one additional silicone, selected from polyorganosiloxanes in the form of oils, waxes, resins or gums.

16: The composition according to claim 15, wherein a content of the additional silicone is from 0.01% to 10% by weight relative to the total weight of the composition.

17: A process for the cosmetic treatment of hair, comprising applying to the hair the composition according to claim 1, and optionally rinsing them with water after an optional leave-on time.

18: The composition of claim 1, wherein the surfactant of formula (I) is selected from the group consisting of tetraalkylammoniumhalides halides in which the alkyl group contains from 12 to 22 carbon atoms, or a mixture thereof.

19: The composition of claim 1 wherein the surfactant of formula (1) is selected from the group consisting of behenyltrimethylammonium chloride, distearyldimethylammonium chloride, cetyltrimethylammonium chloride, benzyldimethylstearylammonium chloride, hydroxyethyl oleyldimethylammonium chloride, palmitylamidopropyltrimethylammonium chloride, stearamidopropyldimethyl(myristyl acetate)ammonium chloride, or a mixture thereof.

20: The composition of claim 1 wherein the fatty alcohol is selected from the group consisting of cetyl alcohol, stearyl alcohol, behenyl alcohol, isocetyl alcohol, isostearyl alcohol, isobehenyl alcohol, oleyl alcohol, and mixtures thereof.
